(19) United States
(12) Patent Application Publication
(10) Pub. No.: US 2021/0186426 A1
(43) Pub. Date: Jun. 24, 2021

(54) SYSTEM AND METHOD FOR DETECTION OF MIDDLE EAR FLUIDS

(71) Applicants: University of Washington, Seattle, WA (US); Seattle Children's Hospital (DBA Seattle Children's Research Institute), Seattle, WA (US)

(72) Inventors: Sharat Raju, Seattle, WA (US); Shyamnath Gollakota, Seattle, WA (US); Justin Chan, Seattle, WA (US); Randall Bly, Seattle, WA (US); Rajalakshmi Nandakumar, Seattle, WA (US)

(73) Assignees: University of Washington, Seattle, WA (US); Seattle Children's Hospital (DBA Seattle Children's Research Institute), Seattle, WA (US)

(21) Appl. No.: 17/274,137

(22) PCT Filed: Sep. 6, 2019
(86) PCT No.: PCT/US19/50083

§ 371 Date: Mar. 5, 2021

Related U.S. Application Data
(60) Provisional application No. 62/728,543, filed on Sep. 7, 2018.

(51) Int. Cl. A61B 5/00; H04M 1/215; H04M 1/03; H04R 1/34; G10L 25/51; H04R 1/02; H04R 1/08; G06N 20/00

(52) U.S. Cl. CPC A61B 5/6817; H04M 1/2155; H04M 1/035; H04R 1/345; G10L 25/51; H04R 1/025; A61B 2562/12; A61B 5/6898; A61B 5/7264; A61B 5/742; G06N 20/00; H04R 2499/11; A61B 2560/0223; H04R 1/08

(57) ABSTRACT

Examples of systems and methods described herein may estimate a state of the ear canal of a patient utilizing a smart phone by characterizing acoustic waveforms reflected off the patient's eardrum. Examples may include an acoustic focusing apparatus that may be connected to a smart phone to provide acoustic signals to and receive reflected signals from an ear canal.

### CROSS-REFERENCE TO RELATED APPLICATIONS

This application claims the benefit under 35 U.S.C. 119 of the earlier filing date of U.S. Provisional Application Ser. No. 62/728,543 filed Sep. 7, 2018, the entire contents of which are hereby incorporated by reference in their entirety for any purpose.

### STATEMENT REGARDING RESEARCH & DEVELOPMENT

This invention was made with government support under Grant No. T32 DC000018, awarded by the National Institutes of Health and Grant No. 1812559, awarded by the National Science Foundation. The government has certain rights in the invention.

### BACKGROUND

The presence of middle ear fluid is a key diagnostic marker for common pediatric ear diseases such as acute otitis media (AOM) and otitis media with effusion (OME). Current methods to detect middle ear fluid, including pneumatic otoscopy and tympanometry, are invasive, expensive or require significant expertise. Pneumatic otoscopy uses puffs of air to examine eardrum mobility under direct visualization and is technically difficult. This procedure is used by only 7-33% of primary care providers. Tympanomtery necessitates a referral to an audiologist and the use of expensive equipment. Therefore, there has long been a need for an accessible, smart phone based solution for detecting middle ear fluid to help caregivers identify and monitor ear fluids and any substance in a patient's ear canal.

Acoustic FMCW signals from smart phones have been used to monitor minute respiratory motions and track finger and hand movements. These designs correlate changes in the reflected FMCW signal to positional changes of a moving object. However, it is impractical to use this technique alone to detect middle ear effusions because sounds waves may reflect off any number of aural structures before entering the ear canal. Such reflections create a highly variable waveform that cannot be used to identify middle ear effusions.

Sometimes, machine learning and algorithms may be used to identify the presence of middle ear fluids. Traditional algorithms transmit an acoustic signal and identify the angle of a dip in the reflection of the acoustic signal. It is understood that a strong reflection, represented by a big dip, is reflective of a blockage in the ear canal. However, merely identifying the angle of the dip to classify a state of the ear canal provides limited accuracy.

### BRIEF SUMMARY

Example methods are disclosed herein. In an embodiment of the disclosure, an example method includes directing an acoustic signal, from a speaker connected to or integral with a smart phone or wearable device, into an ear canal, receiving a reflected waveform responsive to the acoustic signal, at a microphone connected to or integral with the smart phone or wearable device, adjusting the reflected waveform based on the smart phone or wearable device to provide a calibrated waveform, and classifying the calibrated waveform to estimate a state of the ear canal.

Additionally or alternatively, directing a calibration signal, from the speaker, into a calibration environment, and receiving a reflected calibration waveform responsive to the calibration signal at the microphone, and adjusting the reflected waveform comprises using the reflected calibration waveform.

Additionally or alternatively, the acoustic signal comprises a frequency chirp.

Additionally or alternatively, the receiving the reflected waveform comprises receiving the reflected waveform from an eardrum in the ear canal.

Additionally or alternatively, the state of the ear canal comprises an amount of fluid behind an eardrum in the ear canal, a presence of bacteria behind the eardrum, a presence of virus behind the eardrum, a presence of wax in the ear canal, eardrum mobility, or combinations thereof.

Additionally or alternatively, the classifying is based on a shape of the calibrated waveform.

Additionally or alternatively, directing the acoustic signal comprises directing the acoustic signal through an acoustic focusing apparatus coupled to the speaker and wherein receiving the reflected waveform comprises receiving the reflected waveform through the acoustic focusing apparatus.

Example systems are disclosed herein. In an embodiment of the disclosure, a system includes a smart phone, wherein the smart phone comprises a speaker, a microphone, a processor, at least one computer readable media encoded with instructions which when executed, cause the smart phone to perform operations including interrogate an ear canal with an acoustic waveform, from the speaker, receive a reflected acoustic waveform based on the acoustic waveform, at the microphone, create a calibrated waveform based on the reflected acoustic waveform, and classify the calibrated waveform as a state of the ear canal, and an acoustic focusing apparatus coupled to the smart phone to direct the acoustic waveform, from the speaker, into the ear canal, and the reflected acoustic waveform, from the ear canal to the microphone.

Additionally or alternatively, the acoustic focusing apparatus is made of a foldable material.

Additionally or alternatively, the acoustic focusing apparatus is cone-shaped.

Additionally or alternatively, the acoustic focusing apparatus is flattened to conform to an edge of the smart phone having the speaker and the microphone.

Additionally or alternatively, the speaker and the microphone are present in an earbud and wherein the acoustic focusing apparatus is arranged to enclose the speaker at an outer edge of the acoustic focusing apparatus and the microphone at an inner edge of the acoustic focusing apparatus.

Additionally or alternatively, the acoustic focusing apparatus is integrated in a case for the smart phone.

Additionally or alternatively, the acoustic focusing apparatus is clipped onto an edge of the smart phone.

In another aspect of the disclosure, a method includes receiving an acoustic waveform based on an ear canal and a smart phone, detecting a dip in the acoustic waveform, classifying a portion of the acoustic waveform around the dip to provide a probability of a state of the ear canal, and estimating the state of the ear canal based partly on the probability and a threshold associated with the smart phone.

Additionally or alternatively, classifying comprises using a machine learning technique.

Additionally or alternatively, the portion of the acoustic waveform includes a number of points, and wherein the machine learning technique applies a weight to each of the number of points.

Additionally or alternatively, further included is training a model to identify the weight of each of the number of points.

Additionally or alternatively, training the model comprises training the model using a different smart phone than the smart phone used in said receiving the acoustic waveform.

Additionally or alternatively, said receiving and detecting are through an acoustic focusing apparatus coupled to the smart phone.

Additionally or alternatively, the acoustic waveform is a calibrated acoustic waveform based in part on a calibration signal provided by the smart phone, into a calibration environment.

In another aspect of the disclosure, a method includes training a machine learning model using a first computing device to provide a probability of a state of an ear canal based on an acoustic waveform, the training configured to provide a trained model, testing a particular ear canal using a second computing device, different from the first computing device, to obtain a received waveform, and classifying the received waveform using the trained model and a threshold selected in accordance with the second computing device.

Additionally or alternatively, further included is calculating the threshold based on test results from known ear canals using the second computing device.

Additionally or alternatively, the first computing device includes a first model of smart phone, and the second computing device includes a second model of smart phone.

Additionally or alternatively, said training further includes using a first acoustic focusing apparatus, said testing further comprises using a second acoustic focusing apparatus, and said threshold is further selected in accordance with the second acoustic focusing apparatus.

In another aspect of the disclosure, a method includes obtaining a shape of material, the shape of material defining a base end having a size selected to enclose at least one of a speaker or a microphone, a tip end having a size selected for at least partial insertion into an ear canal, and constructing an acoustic focusing apparatus from the shape of material.

Additionally or alternatively, the shape of material comprises a cone.

Additionally or alternatively, the shape of material further defines at least one notch configured to accommodate a housing of the at least one of a speaker or a microphone.

Additionally or alternatively, constructing the acoustic focusing apparatus includes adhering at least a portion of the material to at least another portion of the material.

Additionally or alternatively, obtaining the shape of material includes cutting the shape from a printed template.

In another aspect of the disclosure, a method includes positioning a tip of an acoustic focusing apparatus at least partially into an ear canal, the acoustic focusing apparatus is in acoustic communication with a speaker and a microphone of a computing device, receiving an indication from a sensor of the computing device that the computing device is oriented beyond a threshold angle from horizontal, and displaying, on a display of the computing device, an indication of unacceptable variation from horizontal.

Additionally or alternatively, further included is displaying an indication that the computing device is oriented for measurement of a supine patient.

Additionally or alternatively, further included is displaying a prompt to take a measurement on an upright patient.

Additionally or alternatively, further included is directing an acoustic signal from the speaker into the ear canal, receiving an indication from the sensor of the computing device of movement of the computing device during a time the acoustic signal is provided, and providing an indication to repeat a measurement responsive to the indication of movement.

### BRIEF DESCRIPTION OF THE DRAWINGS

FIG. 1 is a schematic illustration of a system arranged in accordance with examples described herein.

FIG. 2 is a schematic illustration of a system arranged in accordance with examples described herein,

FIG. 3 is a schematic illustration of a system arranged in accordance with examples described herein.

FIG. 4A and FIG. 4B are schematic illustrations of an example smart phone coupled with an example acoustic focusing apparatus in accordance with examples described herein.

FIG. 5 is a schematic illustration of an example earbud coupled with an example acoustic focusing apparatus in accordance with examples described herein.

FIG. 6 is a graphical illustration of example reflected waveforms obtained when an example acoustic signal is played into a patient's ear canal in accordance with examples described herein for both an ear canal with fluid behind the eardrum and without.

FIG. 7 is a graphical illustration of an example calibrated acoustic waveform in accordance with examples described herein.

FIGS. 8A-C are graphical illustrations of examples of cerumen occlusions in accordance with examples described herein.

FIG. 9 is a schematic illustration of a template for an acoustic focusing apparatus arranged in accordance with examples described herein.

FIG. 10 is a schematic illustration of a case with and without a smart phone arranged in accordance with examples described herein.

FIG. 11 is a schematic illustration of a case facilitating use of a pre-existing waveguide, arranged in accordance with examples described herein.

FIG. 12 is a schematic illustration of an example method arranged in accordance with examples described herein.

### DETAILED DESCRIPTION

Certain details are set forth below to provide a sufficient understanding of embodiments of the invention. However, it will be clear to one skilled in the art that embodiments of the invention may be practiced without various of these particular details.

Examples described herein may leverage the ubiquity of smartphones to present an accessible, point-of-care screening tool for middle ear fluid. Examples of systems described herein may operate by (i) sending an acoustic signal (e.g., a soft acoustic chirp) into the ear canal using the smartphone speaker, (ii) detecting a reflected waveform (e.g., reflected sound) from the eardrum using the microphone, and (iii) classifying (e.g., using logistic regression) these reflections to predict a status of the ear canal (e.g., middle ear fluid status). In some examples, no additional hardware (beyond the smartphone) is used beyond an acoustic waveguide, which can be constructed with paper, scissors and tape in some examples. An implemented example system demonstrated equivalent performance while testing across multiple smartphone platforms and when used by untrained adults, highlighting its versatility and ease-of-use.

For example, modulated continuous-wave (FMCW) chirps may be transmitted into a patient's ear canal from a smart phone speaker. The microphone may remain active during the chirp, collecting both incident waves from the speaker and reflected waves from the eardrum. Sound reflected from the eardrum will destructively interfere with the incident chirp and cause feature(s) (e.g., a dip) in sound pressure along a range of frequencies. A normal eardrum resonates well at multiple sound frequencies, creating a broad-spectrum, soft echo; as a result, the shape of the resulting acoustic dip is broad and shallow in the frequency domain. In contrast, an ear canal with a particular state, such as a fluid or pus-filled middle ear, as found in OME and AOM, restricts the vibrational capacity of the eardrum; sound energy that would have vibrated the eardrum is instead reflected back along the ear canal, creating more destructive interference and resulting in differently shaped feature(s) (e.g., narrower and deeper acoustic dip(s)). The acoustic dip generally occurs at the resonant frequency of the ear canal where the quarter-wavelength of the chirp is equal to the length of the canal. Thus, while individual differences in ear canal length affect the location of the dip along the frequency domain, the shape of the feature (e.g., the dip) may primarily depend on the presence or absence of middle ear fluid.

To identify if a patient has middle ear fluid, a raw received waveform may be processed to locate and isolate feature(s) associated with reflections from the eardrum) (e.g., an acoustic dip. Machine learning (e.g., logistic regression) or other techniques may then be used to determine the state of the ear canal associated with the feature(s). For example, whether the shape of the dip is indicative of a normal or fluid-filled ear.

Example systems, methods, and techniques, including implemented examples and data therefrom, are also described in Chan et al., Sci. Transl. Med. 11, eaav1102 (2019) 15 May 2019, which is hereby incorporated by reference in its entirety for any purpose.

Examples described herein may provide a predominantly software-based solution that takes advantage of existing smartphone hardware rather than requiring a separate device. Examples described herein may utilize an acoustic focusing apparatus (e.g., a paper funnel) as a speculum to direct sound into the ear canal. In some examples, the device may be assembled using a printed paper (or other material) template, scissors, and tape. Us of an acoustic focusing apparatus, may advantageously increase reliability of the resulting waveform because sound may be restricted from reflecting off of different structures of the pinnae. Examples described herein utilize classification techniques (e.g., a logistic regression machine learning model) to classify the waveforms received by the microphone.

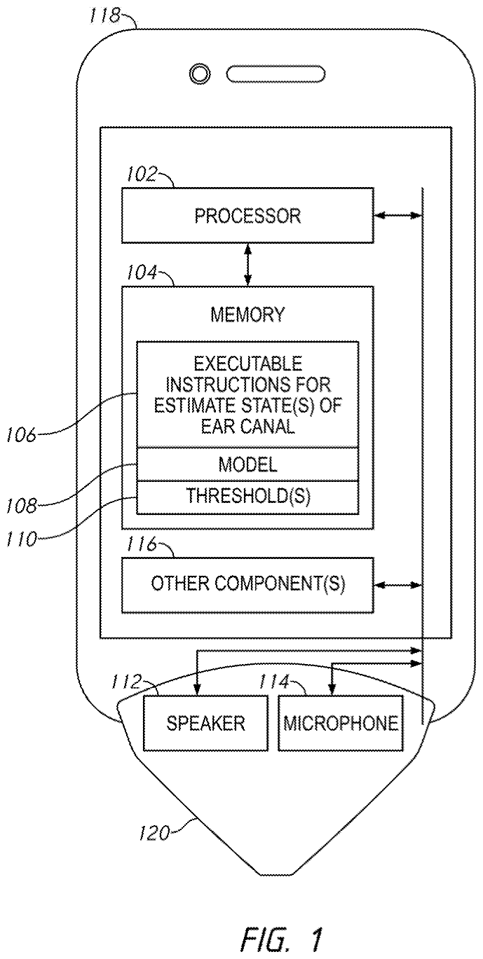

FIG. 1 is a schematic illustration of a system arranged in accordance with examples described herein. The system of FIG. 1 includes a smart phone 118 and acoustic focusing apparatus 120. The smart phone 118 includes processor 102, memory 104, other component(s) 116, speaker 112, and microphone 114. The memory 104 includes executable instructions for estimate state(s) of ear canal 106, model 108, and threshold(s) 110. The components shown in FIG. 1 are exemplary. Additional, fewer, and/or differ components may be used in other examples.

Examples described herein accordingly may include a smart phone, such as smart phone 118. Generally, a smart phone refers to a computing device. The computing device may be handheld, and may have other uses, such as for cellular phone and/or wireless Internet connectivity, although such other use may not be utilized or present in some examples. Examples of smart phones include, but are not limited to, tablets or cellular phones, e.g., iPhones, Samsung Galaxy phones, and Google Pixel phones. Smart phones have become widely available and the ability to estimate state(s) of ear canals using smart phones in accordance with techniques described herein may generally be advantageous in that it may make diagnostics regarding the ear canal more widely available and easily used. The components of the smart phone 118 which may be used in estimating ear canal states (e.g., microphone 114 and/or speaker 112) may not be designed or configured especially for use in that diagnostic application. Moreover, different individual smart phones, types of smart phones, and/or brands of smart phones may have different properties and electronic component responses (e.g., response of the speaker, microphone, and/or processor(s)). Examples described herein may provide systems and techniques which may be utilized to estimate a state of an ear canal notwithstanding variations in the component responses which may be present.

While examples are described herein with reference to smart phones, it is to be understood that techniques described herein may generally be implemented on any computing device in some examples. The computing device used may not have components which were specifically designed and designated for acoustic testing of ears. For example, techniques described herein may be utilized to convert computing device whose primary purpose was not designed to be otologic diagnosis into a device capable of otologic diagnosis. Accordingly, the techniques described herein may be used to adapt the speaker(s), microphone(s), and other components of an existing computing device to be used for analysis of the state of an ear canal. Example computing devices include computers, servers, medical devices. Example computing devices include wearable devices, such as watches, rings, necklaces, pendants, bracelets, hearing aids, smart ear buds, eyeglasses or eyeglass-mounted devices, helmets, and headsets. Generally, computing devices described herein may be Internet-connected devices and/or Bluetooth connected devices with a capability to communicate with other computing devices.

Examples of smart phones described herein, such as smart phone 118, may include one or more speakers, such as speaker 112. The speaker 112 may be in communication with (e.g., electrically connected to) the processor 102. For example, the speaker 112 may be integrated with a device also including the processor 102. In some examples, the speaker 112 may be in wireless communication with a device including the processor 102. The speaker 112 may be used to generate one or more acoustic signals. The speaker 112 may in some examples be integrated with the smart phone 118. In some examples, the speaker 112 may be in electronic communication with the smart phone 118 (e.g., may be connected to the smart phone 118). Examples include when the speaker 112 is provided in an ear bud.

Examples of smart phones described herein, such as smart phone 118, may include one or more microphones, such as microphone 114. The microphone 114 may be in communication with (e.g., electrically connected to) the processor 102. For example, the microphone 114 may be integrated with a device also including the processor 102. In some examples, the microphone 114 may be in wireless communication with a device including the processor 102. The microphone 114 may be used to receive one or more reflected waveforms—such as a reflected acoustic waveform and/or reflected calibration waveform in accordance with techniques described herein. The microphone 114 may in some examples be integrated with the smart phone 118. In some examples, the microphone 114 may be in electronic communication with the smart phone 118. While the speaker 112 and microphone 114 are shown incorporated in a same device, in some examples, the speaker 112 and microphone 114 may be located in separate devices.

In many example smart phones, the speaker 112 and microphone 114 may be generally co-located (e.g., on a same side and/or phase of the smart phone 118). Typically, co-location of the speaker 112 and microphone 114 may be provided to facilitate another function of the smart phone 118, such as voice communication. However, in examples described herein, co-location of the speaker 112 and microphone 114 may be advantageously used to facilitate estimation of state(s) of an ear canal.

Smart phones described herein, such as smart phone 118, may include one or more processors, such as processor 102. Any kind or number of processors may be present, including one or more central processing unit(s) (CPUs), graphics processing unit(s) (GPUs), having any number of cores, controllers, microcontrollers, and/or custom circuitry such as one or more application specific integrated circuits (ASICs) and/or field programmable gate arrays (FPGAs).

Smart phones described herein, such as smart phone 118, may include memory, such as memory 104. Any type or kind of memory may be present (e.g., read only memory (ROM), random access memory (RAM), solid state drive (SSD), secure digital card (SD card)). While a single box is depicted as memory 104, any number of memory devices may be present. The memory 104 may be in communication (e.g., electrically connected) to processor 102.

The memory 104 may store executable instructions for execution by the processor 102, such as executable instructions for estimate state(s) of ear canal 106. In this manner, techniques for estimating state(s) of an ear canal may be implemented herein wholly or partially in software.

The memory 104 may store data which may be used by and/or produced by techniques described herein. For example, the memory 104 may store model 108 and/or threshold(s) 110. While memory 104 is shown as containing executable instructions for estimate state(s) of ear canal 106, memory 104 and threshold(s) 110, those components may be contained on the same memory and/or may be stored on different memories in some examples.

Examples described herein may accordingly provide software for estimating a state of an ear canal. Any number of states of an ear canal may be detected and/or analyzed in accordance with techniques described herein including, but not limited to, presence and/or amount of ear wax in the ear canal, presence and/or amount of fluid in the ear canal (e.g., behind an ear drum), presence and/or amount of organisms in the ear canal (e.g., bacteria and/or viruses behind the ear drum). Examples of states of an ear canal include mobility of an eardrum in the ear canal. Additional examples of states of the ear canal include disease states—e.g., the presence of acute otitis media (AOM) and otitis media with effusion (OME). Although referred to for simplicity as a state of the ear canal, generally, techniques described herein may measure the acoustic impedance of the ear canal, ear drum, middle ear, and inner ear, which can be used for any number of external ear, middle ear, and inner ear diagnoses. Diagnoses include cerumen impaction, OME, AOM, ossicular chain issues, tympanosclerosis, etc. Moreover, techniques described herein may distinguish between these conditions (e.g., by training machine learning models to distinguish between selected conditions). Examples described herein may also be used for acoustic reflex testing, evoked ear potentials, and acoustic emittance.

While the presence and/or amount of ear wax in the ear canal may be a state that may be determined (e.g., estimated) in accordance with techniques described herein, in some examples the presence of ear wax in the ear canal may not impede techniques described herein in estimating another state of the ear canal. This may advantageously allow methods described herein to be performed in ear canals whether or not they include ear wax (e.g., cerumen) or indeed are wholly or partially occluded by ear wax.

Software for estimating a state of an ear canal may include executable instructions for estimate state(s) of ear canal 106. The executable instructions for estimate state(s) of ear canal 106 may include instructions which may cause the smart phone to perform techniques for estimating the state of the ear canal described herein.

Techniques for estimating the state of the ear canal may include interrogating the ear canal with an acoustic waveform (e.g., which may be provided by speaker 112). In some examples, the speaker 112 may be positioned so it is directed toward the ear canal (e.g., by positioning the smart phone 118 to direct the speaker 112 toward an ear canal). The acoustic waveform may generally be an audible waveform. The acoustic waveform may include frequency-modulated continuous-wave (FMCW) chirps. The FMCW chirps may cover a range of frequencies, such as 1.8-4.4 kHz in some examples, 2-4 kHz in some examples, 1.5-3 kHz in some examples, or other example chirp ranges may be used in other examples.

Techniques for estimating the state of the ear canal may include receiving a reflected acoustic waveform (e.g., at microphone 114). The reflected acoustic waveform may be generated by all or a portion of the acoustic waveform provided by speaker 112 being reflected and/or scattered off an ear drum in the ear canal. For example, during all or a portion of time that the acoustic waveform is being provided by the speaker 112, the microphone 114 may remain active and may receive both incident signals from the speaker 112 and reflected signals from the eardrum. Generally, sound (e.g., acoustic waveform(s)) reflected from the eardrum will destructively interfere with the incident acoustic waveform (e.g., chirp) and cause a dip in sound pressure along a range of frequencies. A normal eardrum resonates well at multiple sound frequencies, creating a broad-spectrum, soft echo; as a result, the shape of the resulting acoustic dip is generally broad and shallow in the frequency domain. In contrast, a fluid or pus-filled middle ear, as found in OME and AOM, restricts the vibrational capacity of the eardrum; sound energy that would have vibrated the eardrum is instead reflected back along the ear canal, creating more destructive interference and resulting in differently shaped change in the signal (e.g., a narrower and deeper acoustic dip). The change (e.g., the acoustic dip) generally occurs at the resonant frequency of the ear canal where the quarter-wavelength of the chirp is equal to the length of the canal. Thus, while individual differences in ear canal length may affect the location of the dip along the frequency domain, the shape of the dip primarily depends on the state of the ear canal (e.g., the presence or absence of middle ear fluid).

Techniques for estimating the state of the ear canal may include creating a calibrated waveform based on the reflected acoustic waveform. For example, the processor 102 operating in accordance with executable instructions for estimate state(s) of ear canal 106 may adjust a received reflected acoustic waveform in accordance with a calibration signal to provide the calibrated waveform. This calibration procedure generally may be used to compensate for an open air behavior of the smart phone 18.

Techniques for estimating the state of the ear canal may include classifying the calibrated waveform as a state of the ear canal. For example, the executable instructions for estimate state(s) of ear canal 106 and/or processor 102 may implement a machine learning technique (e.g., regression) which may classify the calibrated waveform as a particular state. The machine learning technique may utilize one or more models (e.g., model 108) to perform the classification. The model 108 may be generated, for example, by training using the smart phone 118 or another device. Training may be implemented, for example, using waveforms generated in ear canals known to have a particular state.

In some examples, models may be trained for use with machine learning techniques described herein. A testing device used to implement the machine learning model and classify a waveform from a patient may be different than one or more of the device(s) used to train the machine learning model. For example, training may be conducted using one configuration of device (e.g., an iPhone) while testing may take place using another configuration of device (e.g., a Samsung Galaxy phone) executing a machine learning technique based on the model trained up using the training device(s). Moreover, testing may be conducted using one configuration of acoustic focusing device while testing may take place using another configuration of acoustic focusing device. One or more thresholds may be used to interpret and/or adjust an output of a machine learning model for a computing device and/or acoustic focusing apparatus which was not used to conduct the training.

Generally, the classification may be based on a shape of all or a portion of the calibrated waveform. In some examples, a feature may be identified in the calibrated waveform (e.g., a dip). Data regarding the waveform at multiple frequencies of the feature may be used for classification. In this manner, an overall shape of the feature (e.g., the dip) may be used for classification, rather than a single metric such as a height and/or angle of the feature. In some examples, sound intensities (e.g., in decibels) for each frequency along an acoustic feature (e.g., an acoustic dip) may be used as separate features as input to a machine learning technique (e.g., logistic regression). Classification may be performed to associate a calibrated waveform with one or more states of an ear canal (e.g., to estimate the state of the ear canal). The classification may compare the calibrated waveform with known information about various ear canal states (e.g., models generated from training data). For example, weights or other parameters for a machine learning classification may be available to classify a calibrated waveform into any of a variety of ear canal states. For example, acute otitis media (AOM) (e.g., presence of infected fluid with pus) may be detected based on a presence of a deeper dip in ear canals having AOM than having otitis media with effusion (e.g., uninfected fluid). Accordingly, classification based on a depth of an acoustic dip or other feature may be able to discriminate as between infected and uninfected fluid behind an eardrum.

In some examples, a different smart phone or device type may be used for testing with a model trained based on a different smart phone and/or device type. A testing approach may be used to support the different testing device, which may avoid and/or reduce a need to collect training data (e.g., clinical data) for every new smartphone or device that may utilize techniques described herein. For example, training data may be used to train a model for a trained smart phone or other computing device. This trained model may be nonetheless used to support future devices of the same or different type. Additionally or instead, training data may be used to train a model for a trained smart phone using a trained acoustic focusing apparatus. This trained model may be nonetheless used to support future devices using the same or different acoustic focusing apparatuses. One or more thresholds may be identified and used to adjust and/or map a probability or other output of the machine learning technique to an estimate of an ear canal state based on the computing device and/or acoustic focusing apparatus used to obtain the measurement.

In some examples, when a new testing device is desired (e.g., different in kind or type or otherwise from the device used to perform the training), the classification technique using the trained model may be tested using the testing device on a number of known targets (e.g., negative ears and/or positive controls). The test may be performed a set number of times each with a set number of waveguide instances, assembled from the same template design in some examples. The waveforms may be passed through the trained model. A set of unscaled probability values may be obtained for every test performed in a negative ear and positive control.

A check may be performed to ensure the probabilities produced for the negative ear do not overlap with the probabilities produced for the positive control. A set of threshold values may then be provided for a given testing device from these unscaled probabilities. There are several possible ways to select a threshold value. One such method would be the largest probability value produced by the negative ears. Another method would be the lowest probability value produced by the positive control. Yet another method would be the median value between the largest probability value produced by the negative ears and the lowest probability value produced by the positive control. The exact method of threshold determination can vary.

Unscaled probability values below this threshold corresponds to a negative prediction, while being equal to or above this threshold corresponds to a positive prediction. During internal testing, this threshold determination method may be used to come up with a threshold for a particular device, e.g., the Samsung Galaxy S6, based on cross-validated data using another device, e.g., the iPhone 5s. With this method, in an implemented example, the clinical accuracies on the Samsung Galaxy S6 on 98 ears were comparable to that produced on the iPhone 5s.

In some examples, accordingly, the classification may be further based on one or more thresholds. The thresholds may be associated with a particular brand, type, and/or model of smartphone used and/or components of the smart phone used. For example, particular smart phones may vary in their response to an ear canal of a given state. Thresholds may be used to compensate for differences between smart phones and/or smart phone components. For example, a machine learning technique described herein may generate a percentage numerical estimate that an ear canal may have a particular state based on a model or other machine learning information. A threshold may be used to determine what percentage is sufficient for the particular smart phone to identify the ear canal as having the particular state. Smart phones may be tested to determine an appropriate threshold for the smart phone.

With unscaled probabilities, an example numerical threshold could be 0.01. However, this does not mean that the likelihood that someone has middle ear fluid is necessarily 1%. The probabilities may be scaled to an index that may more accurately capture the notion of probability of middle ear fluid.

In some examples, the classification technique initially produces an unscaled probability value between 0 and 1. A value below or above the smartphone-specific threshold is a negative or positive result for fluid, respectively. This unsaled value may then be transformed to an index that is reflective of the likelihood of a state of the ear canal (e.g., middle ear fluid status). To do this, the smartphone-specific threshold is “mapped” to the threshold determined when cross-validating the technique across known ears. That is, the threshold determined when initially training a model.

For example, suppose the cross-validation threshold was 0.25 and the new smartphone-specific threshold was 0.05. Unscaled values in the range [0.00, 0.05] may be scaled to [0.00, 0.25] and unscaled values in the range [0.06, 1.00] may be scaled to [0.26, 1.00]. This scaled value may be referred to as the “Middle Ear Fluid Index”.

Smart phones described herein may include any number of other components, such as other component(s) 116 shown in FIG. 1. Examples of other components include one or more displays, user interfaces, networking interfaces, communication interfaces, etc. A display of the smart phone may, for example, be used to display instructions for performing techniques described herein (e.g., play an instructional video and/or instructional graphics regarding how to assemble an acoustic focusing apparatus and/or obtain calibration and/or testing signals described herein) and/or an estimate of an ear canal state determined using techniques described herein. A communication and/or networking interface of the smart phone may be used, for example, to transmit data pertaining to models, thresholds, estimated states, and/or other data described herein. The data may be transmitted to one or more other computing devices, for example. In some examples, smart phones described herein may include one or more positional sensors—including, but not limited to, accelerometer(s), gyroscope(s), geomagnetic sensor(s), inertial sensor(s), and/or GPS device(s).

Examples of systems described herein may include an acoustic focusing apparatus, such as acoustic focusing apparatus 120. Acoustic focusing apparatuses may also be referred to as waveguides. The acoustic focusing apparatus 120 may be positioned to direct an acoustic waveform provided by the speaker 112 from the speaker 112 into an ear canal. The acoustic focusing apparatus 120 may further be positioned to direct a reflected acoustic waveform from the ear canal to the microphone 114. The acoustic focusing apparatus 120 may be coupled to the smart phone 118 (e.g., taped, adhered, clipped, connected). In some examples, the acoustic focusing apparatus 120 may be integrated into a case for the smart phone 118. The acoustic focusing apparatus 120 may generally be sized to enclose the speaker 112 and the microphone 114.

The acoustic focusing apparatus 120 may be cone-shaped, and may be a flattened cone shape in some examples which may conform to an edge of smart phone 118. Other shapes may also be used including cylinders (e.g., pipes), polygonal bases or tips (e.g., squares, rectangles, parallelograms, rhombuses, triangles, pentagons, hexagons etc.), or any other geometric shape (e.g. stars, crosses, crescents, clovers etc.).

In some examples, one or more acoustic coupling devices (e.g., tubes) may be used to acoustically couple a speaker and/or microphone. For example, a tube may be provided between a speaker and a microphone (of a same or different device). The tube may be used as an acoustic focusing device described herein and/or may be acoustically coupled to an acoustic focusing device described herein. In some examples, one or more tubes may be used to couple sound from a speaker to an ear canal and direct sound from the ear canal to a microphone.

Generally, an acoustic focusing apparatus described herein may have a base, a tip, a length, and optional notches. The width of the base can vary, and may generally be sized to surround a speaker, a microphone, or both. In some examples, a width of the base may also span an entire base or surface of a smart phone or other computing device, and may exceed the distance between the speaker and microphone. The tip of the acoustic focusing apparatus may generally be sized to be wholly and/or partially inserted into an ear canal and/or over an ear. Generally, any sized tip may be used. In some examples, a length of the waveguide may vary, and generally any length may be used. Notches may optionally be provided to allow the waveguide to fit snugly with the smart phone or other computing device used.

In some examples, an acoustic focusing apparatus may be wholly or partially implemented using pipe(s)(or other shapes). For example, rubber tubing (or other materials) may be coupled a microphone and/or speaker connected to a computing device. In some examples, a rubber tube may be inserted into a piece of rubber, foam, or other material. The material may be placed over the microphone and/or speaker connected to a computing device. The material may be held in place to the device with any of a variety of materials including, but not limited to, elastic bands, tape, or glue.

An end of the cone may have a diameter sized to approximate an anticipated size on an ear canal opening while being sufficiently large to allow acoustic signals to pass into the ear canal (e.g., 5 mm in some examples, 10 mm in some examples, 15 mm in some examples, 13 mm in some examples, 18 mm in some examples, and other dimensions may be used). Another end of the acoustic focusing apparatus may be sized to enclose a microphone and a speaker of the smart phone. Advantageously, the microphone and speaker of many smart phones may be co-located (e.g., because close placement of these components facilitates noise cancellation during voice communication). The base of the acoustic focusing apparatus cone may vary in size depending on the proximity of the speaker and microphone. For example, in Samsung Galaxy phones, the speaker and microphone are approximately 5 mm apart, such that a smaller conical base may be used. In contrast, they are further apart in iPhones, such that a slightly larger conical base may be used. In one example, an acoustic focusing apparatus may have a base diameter of 90 mm for the Samsung Galaxy S6 and S7, 105 mm for the iPhone 5s, and 115 mm for iPhone 6s and Google Pixel. For each template, the acoustic focusing apparatus was sized with a 15 mm diameter opening which approximates the size of the opening into the ear canal.

The acoustic focusing apparatus may be made from any of a variety of materials. In some examples, the acoustic focusing apparatus may be made from paper (e.g., filler paper, inkjet paper, laserjet paper, cardstock). In some examples, the acoustic focusing apparatus may be made wholly or partially of silicone, plastic, metal, fabric, rubber, glass, aluminum, wood, or concrete. The acoustic focusing apparatus may be disposable in some examples. The acoustic focusing apparatus may not be disposable in some examples.

The acoustic focusing apparatus 120 may be made of a foldable material in some examples, such as paper and/or plastic. In some examples speaker 112 and/or microphone 114 may be present in an ear bud connected to the smart phone 118 and the acoustic focusing apparatus 120 may be arranged to enclose the ear bud, such as by enclosing a speaker 112 at an outer edge of the acoustic focusing apparatus 120 and the microphone 114 at an inner edge of the acoustic focusing apparatus 120. In some examples, the smart phone 202 may include a handheld communication device or a mobile device, or some auxiliary speaker and microphone such as in a wired headset or a wireless headset.

In some examples, the acoustic focusing apparatus may be generally low-cost and simple to generate by a potential user of the system. For example, the acoustic focusing apparatus may be implemented using a conical paper waveguide which may be cut from a printed paper template.

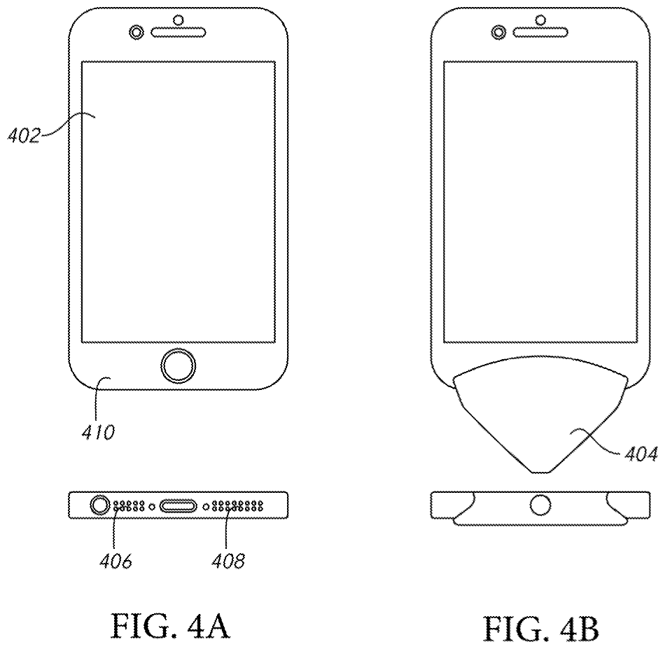

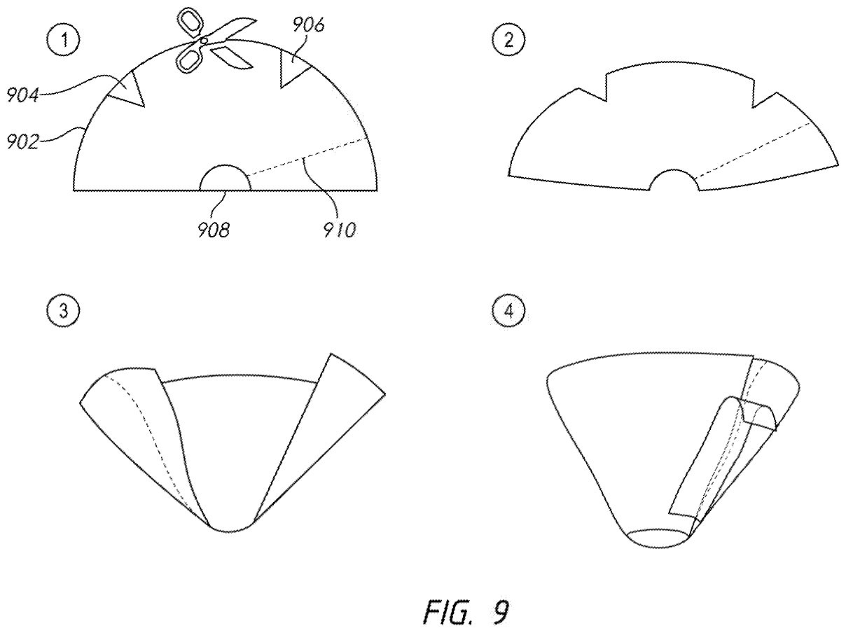

FIG. 9 is a schematic illustration of a template for an acoustic focusing apparatus arranged in accordance with examples described herein. The template 902 includes an outline for cutting out the apparatus. The template 902 has generally a hemispherical shape which may be folded into a cone, so as to form, for example, acoustic focusing apparatus 404 of FIG. 4. The template 902 includes notch 904, notch 906, canal opening 908, and overlap indicator 910. Generally, the template may be printed and/or provided on any of a variety of materials including paper, plastic, and/or metal.

The notch 904 and notch 906 may generally be provided and sized to accommodate portions (e.g., corners) of a smartphone or other device housing adjacent a microphone and/or speaker to be enclosed by the acoustic focusing device.

The canal opening 908 may be sized for insertion into an ear canal, such as using a diameter of between 5 and 10 mm in some examples. The overlap indicator 910 may provide an indicator (e.g., a dashed line, arrows, or other indicator) of where to adhere an edge of the template to assemble a flattened cone apparatus.

To assemble an acoustic focusing apparatus, a flat template may be cut, as shown in the first illustration of FIG. 9. The template would be cut along the solid lines, for example. The cut out template is shown in the next illustration of FIG. 9. The cut template may then be rounded and/or folded into a conical shape as shown in the next illustration of FIG. 9. The ends of the cut template may be positioned to overlap in a region bordered by the overlap indicator 910. The ends of the cut template may then be adhered to one another, as shown in the next illustration of FIG. 9. The ends may be adhered by any of a variety of mechanisms—including tape, adhesive, glue, staples, clips, slotted assembly, etc.

Any of a variety of manufacturing methods may be used to create the acoustic focusing apparatus including any type of printing, cutting, and adhering. In some examples, acoustic focusing apparatuses described herein may be injection molded and/or 3D printed.

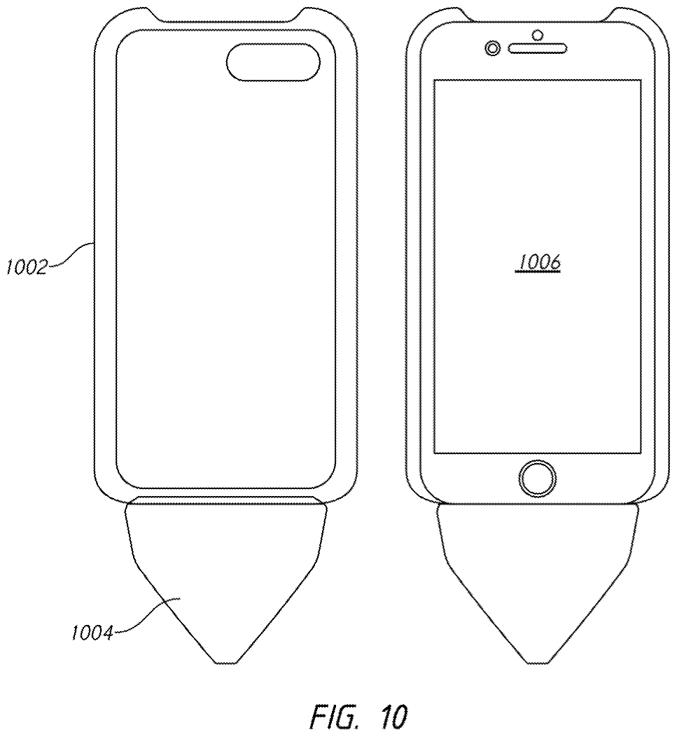

FIG. 10 is a schematic illustration of a case with and without a smart phone arranged in accordance with examples described herein. The case 1002 may be made of any of a variety of materials, such as silicone, and may be sized to enclose a smart phone. The 1002 may be integrated with an acoustic focusing apparatus 1004. The acoustic focusing apparatus 1004 may be made of a same or different material than the remainder of the case 1002 and may be made of generally any material, including those described herein. The acoustic focusing apparatus 1004 may be positioned to enclose a speaker and/or a microphone of the device to be placed in the case 1002. FIG. 10 contains a second view showing a smart phone 1006 placed in the case 1002. The acoustic focusing apparatus may be detachable or not detachable from the case 1002.

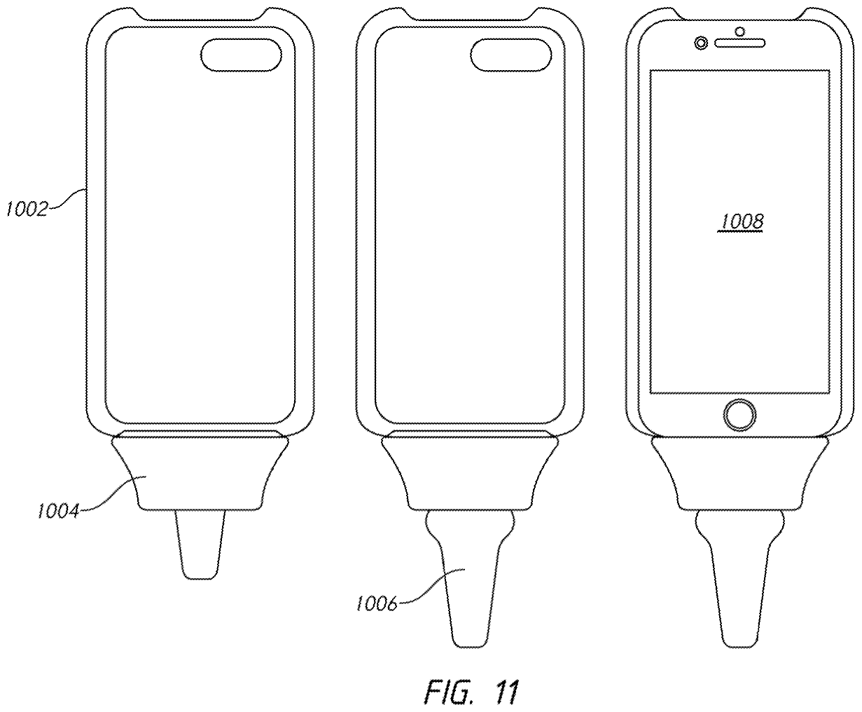

FIG. 11 is a schematic illustration of a case facilitating use of a pre-existing waveguide, arranged in accordance with examples described herein. In some examples, an acoustic focusing apparatus may be implemented using an attachment which may accept a pre-existing waveguide and place the pre-existing waveguide in acoustic communication with a speaker and/or microphone connected to a smart phone or other computing device. The pre-existing waveguide may be, for example, tubing and/or an otoscope specula. In the example of FIG. 11, a case 1102 is shown with an attachment 1104. The attachment 1104 may be integral to the case 1102 and/or may be detachable. The attachment 1104 may be formed from generally any material, including those described herein, and may be positioned to enclose a microphone and/or speaker of a device positioned in the case 1102. Accordingly, a First end of the attachment 1104 may be positioned to receive a speaker and/or microphone. Another end of the attachment 1104 may be positioned and sized to receive a pre-existing waveguide (e.g., an otoscope specula). The waveguide 1106 shown in FIG. 11 may be positioned in the attachment 1104. In this manner, the waveguide 1106 may be acoustically coupled to a speaker and/or microphone of a computing device positioned in the case 1102. FIG. 11 further illustrates a smart phone 1108 positioned in the case 1102.

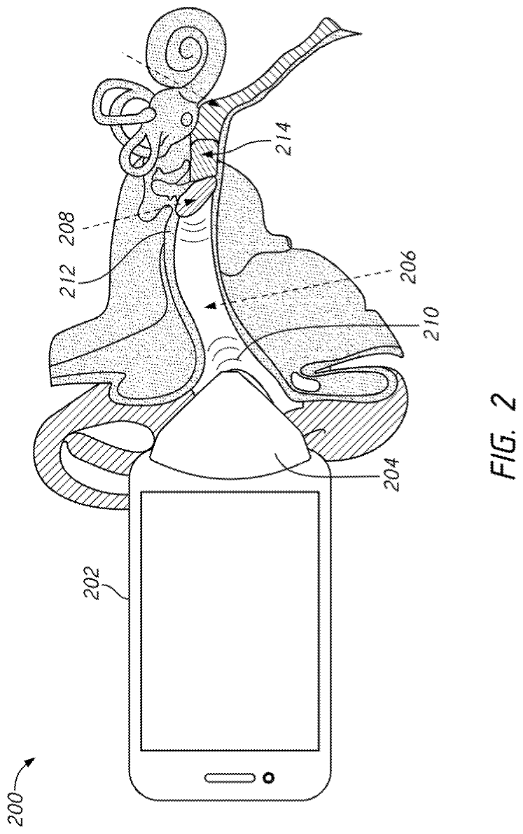

FIG. 2 is a schematic illustration of a system arranged in accordance with examples described herein. In the example of FIG. 2, the system is shown in use in providing and receiving signals from an ear canal. The system 200 included smart phone 202, acoustic focusing apparatus 204, and ear canal 206. The ear canal 206 may include eardrum 208 and there may be fluid 214 present behind eardrum 208. During operation, the smart phone 202 may deliver acoustic waveform 210 into the ear canal 206 and may receive reflected waveform 212 reflected from the eardrum 208. The state of the ear canal (e.g., the presence and/or absence of fluid 214) may produce characteristic features in signals received at the smart phone 202. The smart phone 118 of FIG. 1 may be used to implement smart phone 202 of FIG. 2. The acoustic focusing apparatus 120 of FIG. 1 may be used to implement acoustic focusing apparatus 204 of FIG. 2. The components shown in FIG. 2 are exemplary. Additional, fewer, and/or different components may be used in other examples.

During operation, the acoustic focusing apparatus 204 may be coupled to the smart phone 202. For example, the acoustic focusing apparatus 204 may be clipped, adhered, or otherwise attached to smart phone 202 in a manner which encases a microphone and speaker of the smart phone 202.

The tip of the acoustic focusing apparatus 204 may be positioned at the entrance of (e.g., into) the patient's ear canal 206. In some examples, the acoustic focusing apparatus 204 may point medially and slightly anteriorly into the ear canal 206. In some cases, the ear canal 206 may be straightened during testing by gently pulling the pinnae posteriorly. In some examples, one or more positional sensor(s) on the smart phone 202 may be used to aid in positioning of the acoustic focusing apparatus 204 into the ear canal 206. For example, the smart phone 202 may display an indication when the smart phone is oriented off of horizontal. For example, the smart phone 202 may display a number of degrees titled from horizontal the smartphone may be (e.g., 5°, 10°, etc.). The display may indicate an unacceptable variation from horizontal when the deviation is greater than a threshold (e.g., 45° in some examples, 30° in some examples). The display may indicate that the smartphone may be oriented so as to be inserted into the ear of a supine patient rather than an upright patient. The display, or other output of the smartphone, may prompt a user to change to a measurement of an upright patient, such as by playing recorded speech instructions, or displaying instructions to a user. Accordingly, the smart phone 202 may be positioned such that the speaker is oriented horizontally or within 45° of horizontal in some examples, within 30° in other examples, and other angles may be used. During operation, the smart phone 202 may direct an acoustic waveform 210, such as one or more frequency chirps, from a speaker into the patient's ear canal 206. In some examples, the smart phone 202 may direct the acoustic waveform 210 into the patient's ear canal 206 through the acoustic focusing apparatus 204. In some examples, the acoustic waveform 210 may include audible, 150 ms modulated continuous-wave (FMCW) chirps from 2.0-2.8 kHz. In some examples, the acoustic waveform 210 may be from 2.3-3.8 kHz. In some examples, the acoustic waveform 210 may be from 1.84.4 kHz. In some examples, the acoustic waveform 210 may be played for a duration of 20, 50, 100, 200, 250, 300, 350, or 400 ms. Other durations may also be used. Multiple chirps may be played during the duration, including repeating the chirps over a same frequency range or other frequency ranges. A sequence of 10 chirps may be played in some examples, with other numbers of repetition used in other examples. In one example, 10 identical chirps may be provided with a frequency range of 1.8 kHz to 4.4 kHz, each for a duration of 150 ms. Each chirp may be interspersed with a time of silence, 250 ms of silence in one example, although other amounts may also be used. In some examples, the acoustic waveform 210 may imitate the sound of a bird to create a calming effect for the patient. While noises within chirp frequencies may impact accuracy of the results, ambient noises may typically be outside the chirp frequencies. For example, an infant crying may reach only 400 Hz. In some examples, crying or other unwanted noises may be wholly and/or partially cancelled using components such as low pass filters, high pass filters, band pass filters as well as adaptive filters such as least mean squares filters. Therefore, the crying noise may not impact the techniques described herein.

In some examples, readings from one or more sensors of the smartphone (e.g., accelerometer, gyroscope, and/or geomagnetic sensors), may indicate significant movement or change in orientation of the smartphone during the course of several chirps during the measurement (e.g., due to the patient head or body moving too much or the smartphone moving too much). In some examples, the smartphone may be programmed to provide an indication to the user to repeat a measurement if an amount of movement during the measurement was detected above a threshold.

A microphone of the smart phone 202 may receive a reflected waveform 212 responsive to the acoustic waveform 210 reflected from the patient's eardrum 208. For example, the smartphone may, at least partially simultaneously with the providing of the acoustic waveform, record audio from the microphone for a period of time (e.g., 10s in some examples, although other times may also be used). A sampling rate of 48 kHz was used in one example, although other sampling rates may also be used. In some examples, the microphone may receive the reflected waveform 212 through the acoustic focusing apparatus 204 (e.g., through the aperture of the apparatus that may be wholly and/or partially disposed in an ear canal. The reflected waveform 212 may destructively interfere with the incident acoustic signal and may cause features (e.g., a dip in sound pressure) along a range of frequencies. The feature (e.g., acoustic dip) may occur at the resonant frequency of the ear canal where the quarter-wavelength of the acoustic signal is equal to the length of the canal. Therefore, while individual differences in the ear canal 206 between patients may affect the location of the dip along the frequency domain, the shape of the dip primarily reflects the state of the ear canal 206.

Various processing may occur on the reflected waveform 212. For example, cross-correlation may be performed between the reflected waveform 212 and the acoustic signal to find a starting sample of each chirp in the reflected waveform 212. For each chirp, a transform (e.g., a 48,000 point or other resolution Fast Fourier Transform) may be performed to provide a frequency response. In one example, the frequency response may be found from 0-24 kHz, although other frequency ranges may be used. Frequencies outside of the transmitted chirp range (e.g., outside of 1.8-4.4 kHz in some examples) may be discarded. Chirps that were two or more standard deviations from the mean of all recorded chirps may be excluded from further analysis. In some examples, only certain chirps may be analyzed and the remainder excluded.

The reflected waveform 212 (e.g., a combination of the reflected waveform 212 and the incident acoustic waveform 210) may be adjusted based on a calibration to provide a calibrated waveform. The calibration may occur prior to interrogation of a particular ear canal, and may be particular to the smart phone 202 used for interrogation. Generally, the calibration may be used to reduce variations in the received waveform caused by the particular arrangement of the smart phone used and/or environment. For example, a calibration procedure may be used which generates a response of the smart phone components in a calibration environment (e.g., in the absence of an ear canal). A combination of the reflected waveform 212 and signals generated during the calibration process may therefore provide a calibrated waveform which may be more reliably classified, having smart phone specific and/or environment specific features in the waveform reduced and/or eliminated.

In some examples, filtering may be used to smooth the reflected waveform 212 and/or the calibrated waveform. A feature-detection algorithm (e.g., a peak detection algorithm) may be used identify the feature (e.g., acoustic dip) associated with sound waves being reflected off the eardrum. For example, the most prominent features (e.g., acoustic dips) may be identified within a frequency range, such as within 2.3-3.8 kHz in some examples. Frequencies within a range of the frequency of the feature (e.g., within 500 Hz of the feature in some examples) may be utilized for further processing. In this manner, machine learning techniques may be focused on data associated with the portions of the acoustic response most predictive of a state of the ear canal (e.g., middle ear effusion status).

The calibrated waveform may be classified based on a machine learning technique to estimate a state of the ear canal 206 using a shape of the calibrated waveform.

After operation, the smart phone 202 may be withdrawn from the ear and repositioned in approximately the same location to produce a second set of acoustic signals for validation. This process may be repeated for each operation.

An estimated state of the ear canal may be displayed to a user (e.g., on a display of the smart phone 202). For example, a display may indicate that fluid is present in the ear canal. A display may indicate that bacteria is present in the ear canal. A display may indicate that a viral load is present in the ear canal. The estimated state of the ear canal may additionally or instead be stored (e.g., in a memory accessible to the smart phone 202) and/or may be transmitted to another computing device (e.g., to a computing device accessible to a healthcare provider). In some examples, a text-based message may be presented on a display to the user indicating a result: e.g., “suggestive of middle ear fluid” or “middle ear fluid unlikely”.

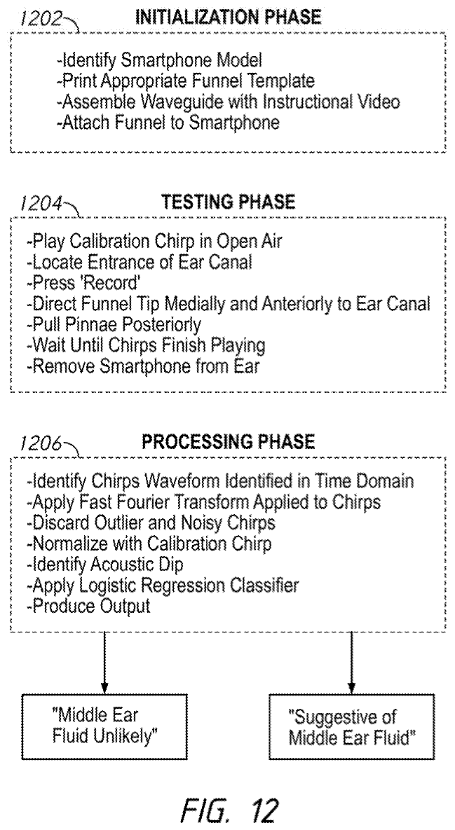

FIG. 12 is a schematic illustration of an example method arranged in accordance with examples described herein. In some examples, various actions may be performed during an initialization phase to prepare for testing a patient. For example, in the initialization phase 1202 of FIG. 12, a smartphone model may be identified (e.g., a brand, model of device and/or a brand and/or model of speaker and/or microphone). A template for an acoustic focusing apparatus (e.g., a funnel) may be printed and/or obtained. The acoustic focusing apparatus (e.g., waveguide) may be assembled. In some examples, the acoustic focusing apparatus may be assembled using instructions, which may be displayed in some examples by the smart phone to be used for testing. The acoustic focusing apparatus (e.g., funnel) may be attached to the smart phone.

In some examples, various actions may be performed during a testing phase for testing a particular patient. For example, in testing phase 1204, a calibration chirp may be played into a calibration environment (e.g., open air). An entrance to the ear canal may be located, recording may be initiated on the smartphone, and the acoustic focusing apparatus tip (e.g., funnel) may be directed medially and anteriorly into the ear canal. In some examples, the pinnae may be pulled posteriorly to facilitate acoustic access to the ear canal. Acoustic signals (e.g., chirps) may be delivered to the ear canal. During delivery of the acoustic signals, received signals may be recorded by a microphone of the device used for testing. When the acoustic signals are finished playing—which may be indicated, for example, by an indicator on a display of the smart phone and/or sound played by the smart phone—the acoustic focusing apparatus (which may be connected to the smart phone) may be removed from the ear canal.

In some examples, various actions may be performed during a processing phase to process signals received during testing. For example, in processing phase 1206, chirps in the received acoustic signals may be identified in the time domain and transformed (e.g., using a fast Fourier transform) to the frequency domain. Outlier and/or noisy chirps may be discarded, and the chirps may be normalized using the signals received during calibration (e.g., responsive to the calibration chirp in the calibration environment). An acoustic feature (e.g., acoustic dip) may be identified, and may be classified (e.g., using logistic regression). The classification may in some examples provide a probability of a particular diagnosis (e.g., middle ear fluid). In some examples, a threshold may be used which may be specific to the smartphone model or type which may relate the classification probability to an ultimate estimate of the ear canal state. An output of the estimated ear canal state may be provided—such as “middle ear fluid unlikely” or “suggestive of middle ear fluid.”

The example of FIG. 12 is exemplary only. Other phases may be used in other examples and fewer, additional, and/or different actions may be performed during each depicted phase. The executable instructions for estimate state(s) of ear canal 106 of FIG. 1 may include instructions for performing the delivery of calibration and/or other acoustic chirps during the testing phase 1204 and the actions depicted as occurring during the processing phase 1206.

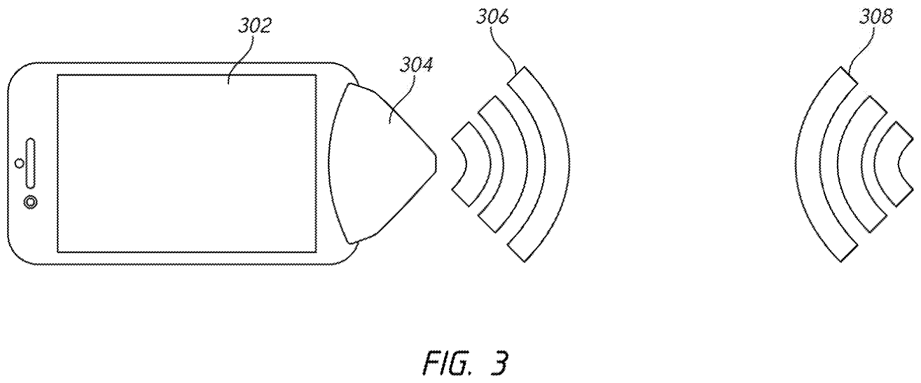

FIG. 3 is a schematic illustration of a system arranged in accordance with examples described herein, FIG. 3 depicts smart phone 302 during a calibration process. The smart phone 302 may be coupled to acoustic focusing apparatus 304, which may enclose a microphone and speaker of the smart phone 302. The smart phone 302 may provide calibration signal 306 and receive reflected calibration waveform 308 from a calibration environment. The smart phone 302 may be implemented using the smart phone 118 of FIG. 1 and/or smart phone 202 of FIG. 2. The acoustic focusing apparatus 304 may be implemented using the acoustic focusing apparatus 120 of FIG. 1 and/or acoustic focusing apparatus 204 of FIG. 2. The components of FIG. 3 are exemplary only, and additional, fewer, and/or different components may be used.

An example calibration process is described with reference to FIG. 3. The calibration process may in some examples be performed using a same smart phone as will be used to provide acoustic signals to an ear canal for estimation of the ear canal state. Moreover, in some examples the calibration process may be performed in an environment similar to the environment in which signals will be provided to an ear canal (e.g., in a same room, building, and/or city). In some examples, the calibration may be performed by a different smart phone than will later use the calibration results to estimate an ear canal state, and data regarding the calibration may be provided to the smart phone used to estimate ear canal state (e.g., by storing calibration data in a location accessible to the smart phone). The calibration may preferably be performed by a smart phone having a same brand, type, or model as the smart phone used to estimate ear canal state.

Generally, calibration may be performed prior to providing acoustic signals to an ear canal for use in estimating an ear canal state. However, in some examples, waveforms may be received from an ear canal, data may be stored regarding the received waveforms, and calibrated in accordance with later-received calibration information. Accordingly, in some examples, a calibration procedure may be performed after providing acoustic signals to an ear canal.

The calibration procedures may be used to reduce the variability caused by different waveguides as well as microphone and speaker differences across smart phones. Calibration may be desirable and/or necessary to improve an ability of a machine learning technique to later classify a resulting calibrated waveform. For example, without calibration, the received waveforms may vary in accordance with particular smart phones such that it may be difficult or impractical to classify them using a trained machine learning technique.

During calibration, the smart phone 302 (e.g., a speaker of the smart phone 302) may direct a calibration signal (e.g., a chirp) into a calibration environment through an acoustic focusing apparatus 304. The calibration signal may be similar (e.g., the same) as an acoustic signal that will be provided to an ear canal. For example, the calibration signal may be an acoustic signal including one or more frequency chirps. the frequency chirps may occur at the same and/or overlapping frequencies to those used to interrogate an ear canal for estimating ear canal state.

The calibration environment may generally be an open air environment (e.g., an environment providing minimal reflected waves). In some examples, the calibration environment may be a known environment for which reflection properties are understood, such as a known ear canal, simulated ear canal (e.g., plastic tube), or other material. The smart phone 302 may receive a reflected calibration waveform 308 responsive to the calibration signal through the acoustic focusing apparatus 304, reflected from the calibration environment. The reflected calibration waveform 308 may be used for calibrating signals received during interrogation of an ear canal. For example, the reflected calibration waveform 308 may be normalized by combining the calibration signal 306 and the reflected calibration waveform 308 to determine a baseline signal. The baseline signal may represent a unit frequency response of the speaker and microphone of the smart phone 302 and the acoustic focusing apparatus 304.

Referring back to FIG. 2, the reflected waveform 212 obtained during testing may be adjusted based on the reflected calibration waveform 308 and/or the baseline signal to provide the calibrated waveform used for classification. For example, the reflected waveform 212 may be scaled and/or combined with the reflected calibration waveform 308 and/or baseline calibration signal 306. In some examples, a set of weights may be generated based on the baseline signal, and the weights may be used to normalize measurements received from a patient's ear canal.

The weights and/or other information about the baseline may be stored, for example in memory 104 of FIG. 1. Instructions for performing calibration and obtaining the baseline signal and/or weights may be included in executable instructions for estimate state(s) of ear canal 106 of FIG. 1 in some examples.

FIG. 4A and FIG. 4B are schematic illustrations of a smart phone coupled to an acoustic focusing apparatus in accordance with examples described herein. FIG. 4A illustrates smart phone 402 have a lower edge 410. A microphone 406 and speaker 408 may be co-located at the edge 410 as shown in the side view having a straight-on view of edge 410. In FIG. 4B, acoustic focusing apparatus 404 is attached to smart phone 402. The front view shows the conical acoustic focusing apparatus 404 coupled to the smart phone 402. The side view having a straight-on view of edge 410 depicts the flattened nature of the acoustic focusing apparatus 404, such that the acoustic focusing apparatus 404 may be conformed to a shape of the edge 410 and encompass the microphone 406 and speaker 408. An opening is provided in the conical shape of acoustic focusing apparatus 404 that may be sized to be wholly or partially inserted in an ear canal.

The smart phone 402 of FIG. 4A and FIG. 4B may be implemented using any smart phone described herein, including smart phone 118 of FIG. 1, smart phone 202 of FIG. 2, and/or smart phone 302 of FIG. 3. The acoustic focusing apparatus 404 may be implemented using the acoustic focusing apparatus 120 of FIG. 1 and/or acoustic focusing apparatus 204 of FIG. 2 or acoustic focusing apparatus 304 of FIG. 3. The components of FIG. 4A and FIG. 4B are exemplary. Additional, fewer, and/or different components may be used in other examples.

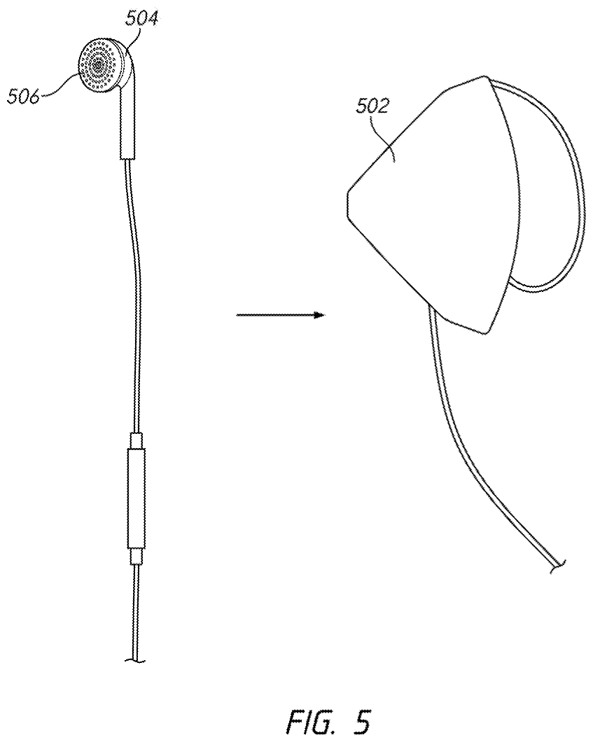

FIG. 5 is a schematic illustration of an example earbud coupled with an example acoustic focusing apparatus in accordance with examples described herein. The example of FIG. 5 illustrates a microphone 504 and speaker 506 of an earbud. The microphone 504 and speaker 506 may be encased by an acoustic focusing apparatus 502. The earbud may be used to implement the speaker 112 and/or microphone 114 of FIG. 1 in some examples. The earbud of FIG. 5 may be used to implement the speaker and microphone of the smart phone 202 of FIG. 2 in some examples. The earbud of FIG. 5 may be used to implement the speaker and microphone of the smart phone 302 of FIG. 3 in some examples.

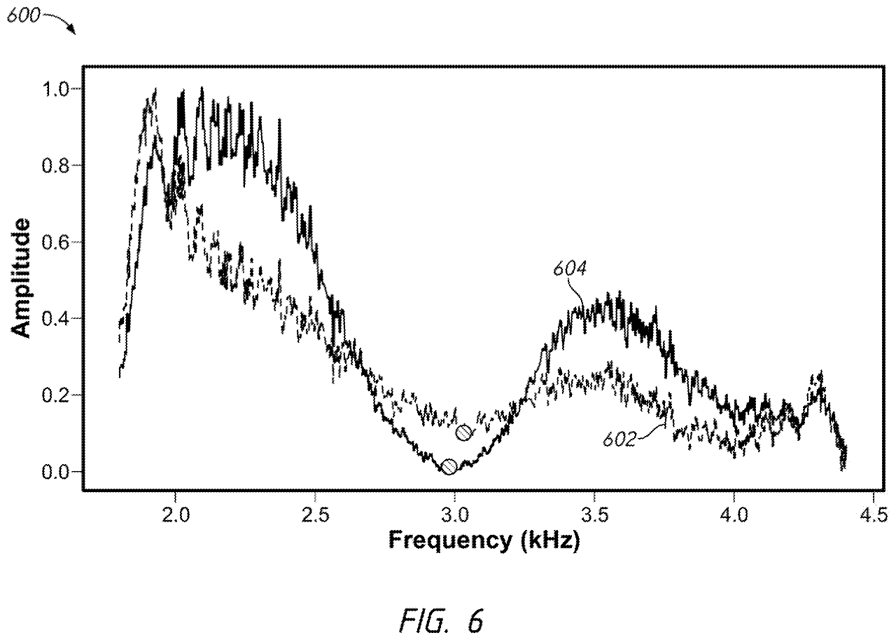

FIG. 6 is a graphical illustration of example reflected waveforms obtained when an example acoustic signal is played into a patient's ear canal in accordance with examples described herein for both an ear canal with fluid behind the eardrum and without. The raw waveform of an eardrum full of fluid 604 has a more prominent acoustic dip. A bottom of the dip is indicated along the raw waveform of an eardrum full of fluid 604 using a prominent dot in FIG. 6. The raw waveform of a normal eardrum 602 with a shallower acoustic dip may occur at a higher frequency due to an effectively shorter canal and corresponding quarter-wavelength. A bottom of the dip is indicated along raw waveform of a normal eardrum 602 using a prominent dot in FIG. 6. Typically, a number of points of data making up the dip may be used for classification. The waveforms of FIG. 6 are provided by way of example of the kinds of changes in reflected waveforms which may be caused by difference ear canal states. It is generally these changes that the techniques and systems described herein may utilize to classify waveforms.

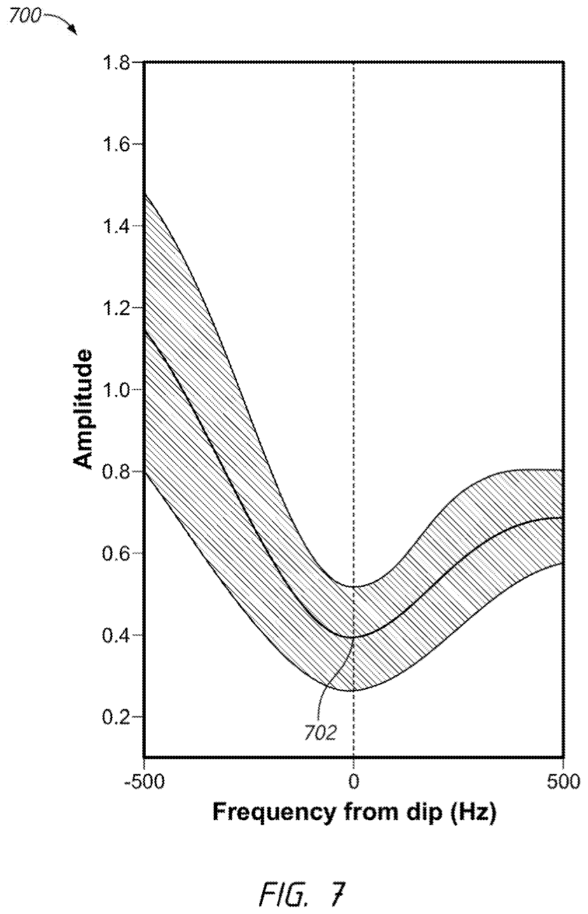

FIG. 7 is a graphical illustration of a calibrated acoustic waveform arranged in accordance with examples described herein. As shown in FIG. 7, the graphical representation of a calibrated acoustic waveform is generally an adjusted (e.g., smoothened) curve as compared to the raw acoustic waveform shown in FIG. 6. The calibrated acoustic waveform may represent an output of a moving average window. For example, a moving average filter with a window size. In some examples, the window size may be 300 samples. Other window sizes may be used including, but not limited to, 100, 150, 200, 250, 350, 400, 450, 500 samples. The filter may be implemented, for example, by any smart phone described herein.

Once the most prominent feature (e.g., dip) within a particular frequency range in the calibrated acoustic waveform is identified, some number of frequency points on either side may be collected and used for classification. For example, 500 points to the left and 500 points to the right of the dip 702 of FIG. 7 may be collected and analyzed. The number of points measured around the dip is not limited to 500. For example, the number can be 100, 200, 400, 800, 1200, 1600, or another number may be used. These point values may form an array where each element may represent the amplitude for each of the selected frequencies around the acoustic feature (e.g., dip 702). Each of the acoustic signals may be aggregated into a single matrix. Using a logistic regression machine learning algorithm, each of the points collected may be assigned a weight to determine if the state of the ear canal. The depth of the acoustic dip may be given the most weight in some examples. Models described herein may specify the weights, such as model 108 of FIG. 1. For example, machine learning techniques described herein may be trained to develop a set of weights—with a weight associated in some examples with each point (e.g., frequency) from a set of points collected which represent an acoustic feature (e.g., acoustic dip). Sound intensities at the top and bottom of the acoustic waveform may be given the most weight by the predictive model. Accordingly, an acoustic pattern may be independently identified for middle ear fluid consistent with the known acoustic response of the eardrum. The model may be trained with data collected from patients or other sources using the respective smart phone and/or a different smart phone.

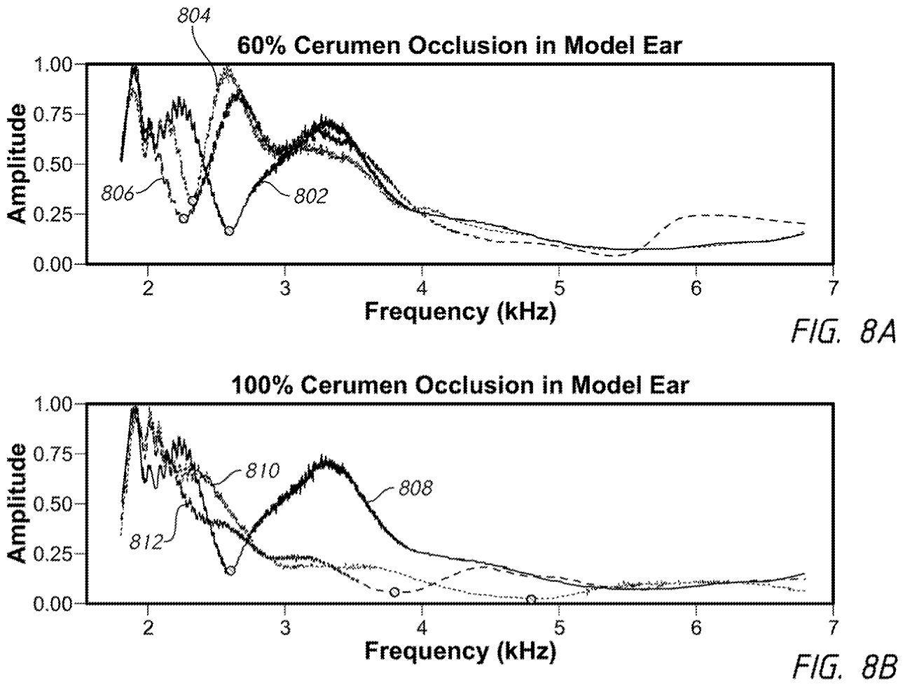

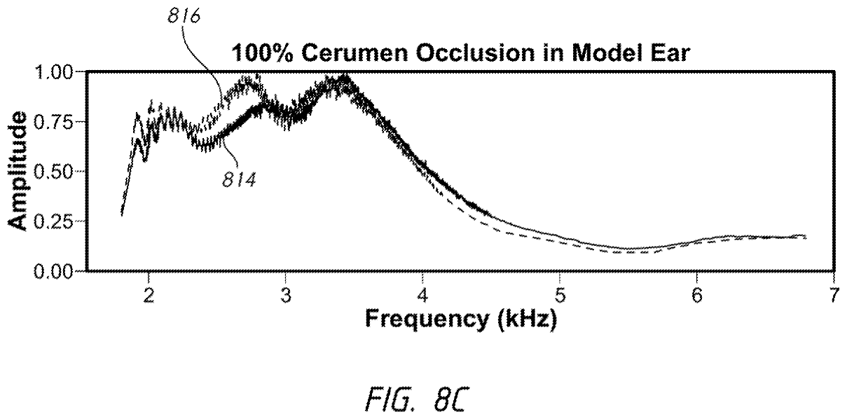

FIGS. 8A-C are graphical illustrations of examples of cerumen occlusions in accordance with examples described herein. Generally, examples described herein may alert users to the presence of cerumen (e.g., ear wax) and/or may accurately estimate a state of an ear canal notwithstanding the presence of cerumen in the canal. The graphs of FIGS. 8A-C illustrate waveforms generated in a model ear utilizing putty to mimic cerumen and demonstrate the type of changes cerumen may make to waveforms described herein. FIG. 8A illustrates a situation where there is partially occluding wax (60-70%) in the model ear. The partial occlusion generally had little effect on the shape or position of the features indicative of the state of the ear canal. For example, waveform 802 was gathered when there was no occlusion in the model ear. Waveform 804 was gathered when there was a 60% occlusion measured at 0 cm deep in the model ear. Waveform 806 was gathered when there was a 60% occlusion measured at 1 cm deep. Generally, a shape and position of a feature (e.g., acoustic dip) may be relatively similar among the three waveforms—waveform 802, waveform 804, and waveform 806. Accordingly, techniques described herein may be insensitive to partial occlusions.

FIG. 8B illustrates a situation where there is 100% cerumen occlusion (e.g., impaction). Generally, as the site of impaction moves closer to the entrance of the ear canal, a relevant feature (e.g., acoustic dip) may appear shallower and occur at a higher frequency due to an effectively shorter canal and corresponding quarter-wavelength. Waveform 808 illustrates an ear with no occlusion. Waveform 810 illustrates an ear with 100% occlusion measured at 0.5 cm deep. Waveform 812 illustrates an ear with 100% measured at 1 cm deep. In these cases, chirps can reflect off cerumen, generating a false feature (e.g., acoustic dip) that may not be representative of middle ear status. For example, at a depth of 1 cm, which is the deepest point cerumen would naturally accumulate, a false acoustic dip is shown in waveform 812 located approximately 1 kHz higher compared to a normal dip from eardrum reflections in waveform 808. At shallower depths of impaction, the false dip was even more right-shifted, as shown in waveform 810. Accordingly, examples described herein may provide an error or alert if a feature (e.g., acoustic dip) is identified at a frequency that is outside of an expected range. For example, an acoustic dip representative of middle ear status may be expected at around 3 kHz in some examples (e.g., between 2.4-3.7 kHz). An alert may be displayed and/or provided when a feature (e.g., acoustic dip) is instead identified outside this range.

Moreover, a shallow cerumen occlusion may generate a reflected waveform similar to that reflected responsive to a calibration signal (e.g., similar to an open air calibration environment). FIG. 8C illustrates this scenario, with waveform 814 corresponding to data generated responsive to a calibration chirp in an open-air calibration environment. The waveform 816 corresponds to data reflected from an ear with 100% occlusion at 0 cm deep. Accordingly, in some examples, systems described herein may provide an alert when a reflected waveform resembles a reflected calibration waveform.

The system of FIG. 1 may be arranged to provide the cerumen-related alerts described herein. For example, the executable instructions for estimate state(s) of ear canal 106 may include instructions for determining whether a feature is outside of an expected range and/or if a reflected waveform resembles a reflected calibration waveform and providing the corresponding alert. The alert may be displayed on the smart phone, may be implemented using an audible tone and/or spoken alert, and/or may be stored and/or transmitted to another computing device

Diagnosis and treatment of middle ear infection currently has necessitated astute clinical decision-making. For example, prescribing antibiotics for acute otitis media may utilize clinical context apart from the presence of middle ear fluid, such as infection severity, time-course, and systemic symptoms. For recurrent infections and persistent effusions, referral to a specialist is recommended for possible surgical management. However, even after initial physician consultation, patients may require repeat visits to monitor middle ear fluid, which can result in high utilization of healthcare systems. Under physician guidance, technology described herein may allow laypersons (e.g., parents or patients) to monitor an ear canal without purchasing additional medical hardware.

### Implemented Example

An implemented example system was used in a 98-patient-ear study including pediatric patients between 18 months and 17 years of age were drawn from two different subgroups: i) patients undergoing ear tube placement, a common surgery performed on patients with chronic OME or recurrent AOM (n=48 ears), and ii) patients undergoing a different surgery, such as tonsillectomy, without recent symptoms of AOM or OME and without signs of middle ear fluid on physical examination (n=50 ears). A receiver-operating characteristic (ROC) curve was generated from the cross-validation step, with an area under the curve (AUC) of 0.865. The operating point was chosen to have an overall sensitivity and specificity of 84.6% (95% CI: 65.1-95.6%) and 80.6% (95% CI: 69.5-88.9%), respectively. With K-fold (K=10) cross-validation, a comparable AUC of 0.847 was obtained.

The algorithm predicted that ears with narrower and deeper acoustic dips were more likely to have middle ear fluid. Similarly, on univariate analysis, sound intensities at the top and bottom of the waveform, which determine the depth of an acoustic dip, were given the most weight by the predictive model). Acoustic reflectometry, which also assesses middle ear fluid status, demonstrated an AUC of 0.774, similar to previously published results. Therefore, the smart phone algorithm's improved clinical performance may be the result of applying machine learning over the waveform rather than relying on a few hand-selected features used by acoustic reflectometers.

Data for the study was collected using both the iPhone 5s and the Samsung Galaxy S6. Specifically, the entire iPhone 5s dataset was used for training except for one patient ear which was “held out” for testing. The trained algorithm was then tested on Galaxy S6 data from the held out ear. This was repeated for all patient ears in the cohort to generate an AUC of 0.858. In the same manner, testing was also performed on a subset of the patient cohort using an iPhone 6s (n=10 ears), Samsung Galaxy S7 (n=12), and Google Pixel (n=8). The algorithm correctly classified 80% (8 of 10) of iPhone 6s, 91.7% (11 of 12) of Galaxy S7, and 87.5% (7 of 8) of Pixel data.

The usability of the acoustic focusing apparatus was tested on 10 untrained adults. The 10 participants were shown a short instructional video and were asked to create and mount an acoustic focusing apparatus using a paper template, tape, and scissors. The average time required to cut, fold, and attach the smartphone-mounted waveguide was 2.8 (±0.89) minutes. Second, participants tested their waveguide on the ear of a subject who had no middle ear effusion. The same subject's ear was used for testing by all participants to ensure consistency of results. Raw acoustic waveforms generated from a single subject's ear were similar for both untrained and trained users. Furthermore, the algorithm correctly classified all curves as not having middle ear effusion. For the overall system, participants gave an average usability rating of 8.9 (±1.0) on a scale of 1 (unusable)-10 (extremely usable).

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein.” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A method comprising:
directing an acoustic signal, from a speaker connected to or integral with a smart phone or wearable device, into an ear canal;
receiving a reflected waveform responsive to the acoustic signal, at a microphone connected to or integral with the smart phone or wearable device;
adjusting the reflected waveform based on the smart phone or wearable device to provide a calibrated waveform; and
classifying the calibrated waveform to estimate a state of the ear canal.

2. The method of claim 1, further comprising:
directing a calibration signal, from the speaker, into a calibration environment; and
receiving a reflected calibration waveform responsive to the calibration signal at the microphone; and wherein, adjusting the reflected waveform comprises using the reflected calibration waveform.

3. The method of claim 1, wherein the acoustic signal comprises a frequency chirp.

4. The method of claim 1, wherein the receiving the reflected waveform comprises receiving the reflected waveform from an eardrum in the ear canal.

5. The method of claim 1, wherein the state of the ear canal comprises an amount of fluid behind an eardrum in the ear canal, a presence of bacteria behind the eardrum, a presence of virus behind the eardrum, a presence of wax in the ear canal, eardrum mobility, or combinations thereof.

6. The method of claim 1, wherein the classifying is based on a shape of the calibrated waveform.

7. The method of claim 1, wherein directing the acoustic signal comprises directing the acoustic signal through an acoustic focusing apparatus coupled to the speaker and wherein receiving the reflected waveform comprises receiving the reflected waveform through the acoustic focusing apparatus.

8. A system comprising:
a smart phone, wherein the smart phone comprises:
a speaker;
a microphone;
a processor;
at least one computer readable media encoded with instructions which when executed, cause the smart phone to perform operations comprising:
interrogate an ear canal with an acoustic waveform, from the speaker;
receive a reflected acoustic waveform based on the acoustic waveform, at the microphone;
create a calibrated waveform based on the reflected acoustic waveform; and
classify the calibrated waveform as a state of the ear canal; and
an acoustic focusing apparatus coupled to the smart phone to direct the acoustic waveform, from the speaker, into the ear canal, and the reflected acoustic waveform, from the ear canal to the microphone.

9. The system of claim 8, wherein the acoustic focusing apparatus is made of a foldable material.

10. The system of claim 8, wherein the acoustic focusing apparatus is cone-shaped.

11. (canceled)

12. The system of claim 8, wherein the speaker and the microphone are present in an earbud and wherein the acoustic focusing apparatus is arranged to enclose the speaker at an outer edge of the acoustic focusing apparatus and the microphone at an inner edge of the acoustic focusing apparatus.

13. The system of claim 8, wherein the acoustic focusing apparatus is integrated in a case for the smart phone.

14. The system of claim 8, wherein the acoustic focusing apparatus is clipped onto an edge of the smart phone.

15. A method comprising:
receiving an acoustic waveform based on an ear canal and a smart phone;
detecting a dip in the acoustic waveform;
classifying a portion of the acoustic waveform around the dip to provide a probability of a state of the ear canal; and
estimating the state of the ear canal based partly on the probability and a threshold associated with the smart phone.

16. The method of claim 15, wherein classifying comprises using a machine learning technique.

17. The method of claim 16, wherein the portion of the acoustic waveform comprises a number of points, and wherein the machine learning technique applies a weight to each of the number of points.

18. The method of claim 17, further comprising training a model to identify the weight of each of the number of points.

19. The method of claim 18, wherein training the model comprises training the model using a different smart phone than the smart phone used in said receiving the acoustic waveform.

20. The method of claim 15, wherein said receiving and detecting are through an acoustic focusing apparatus coupled to the smart phone.

21. The method of claim 15, wherein the acoustic waveform is a calibrated acoustic waveform based in part on a calibration signal provided by the smart phone, into a calibration environment.

22-34. (canceled)
